(19) United States
(12) Patent Application Publication
(10) Pub. No.: US 2008/0092903 A1
(43) Pub. Date: Apr. 24, 2008

(54) Laryngeal mask assemblies

(75) Inventor: John Edward Nash, Hythe (GB)

Correspondence Address: LOUIS WOO;LAW OFFICE OF LOUIS WOO, 717 NORTH FAYETTE STREET, ALEXANDRIA, VA, 22314, US

(73) Assignee: Smiths Group plc, London (GB)

(21) Appl. No.: 11/907,351

(22) Filed: Oct. 11, 2007

(30) Foreign Application Priority Data
Oct. 20, 2006 (GB) 0620862.3

(51) Int. Cl. A61M 16/06

(52) U.S. Cl. 128/207.15

(57) ABSTRACT

A laryngeal mask has an inflatable annular sealing cuff extending around a mount at the patient end of a tube. The mask also has an annular collar on the cuff extending around its opening. The collar is triangular in section with an inflated height of about 10 mm and is soft and floppy when not inflated. The cuff and collar are separately inflatable via separate inflation lines.

### BACKGROUND OF THE INVENTION

This invention relates to laryngeal mask assemblies.

It is common practice to use an airway known as a laryngeal mask for administering anaesthetic and ventilation gases to a patient. These airways comprise a tube with an inflatable mask or cuff at one end, the tube being inserted in the patient's mouth so that one end is located in the hypopharynx and so that the mask forms a seal in this region with the surrounding tissue. Laryngeal masks are described in, for example, U.S. Pat. No. 5,355,879, U.S. Pat. No. 5,305,743, U.S. Pat. No. 5,297,547, U.S. Pat. No. 5,282,464, GB2267034, U.S. Pat. No. 5,249,571, U.S. Pat. No. 5,241,956, U.S. Pat. No. 5,303,697, GB2249959, GB2111394, EP448878, U.S. Pat. No. 4,995,388, GB2205499, GB2128561, GB2298797, GB2334215, GB2337020, PCT/GB00/03044, WO01/13980, GB0002805, GB0020274, GB2371990 and GB2409163. Laryngeal masks usually comprise a curved, extruded shaft, a separate mount member joined at the patient end of the tube and an inflatable cuff attached to the mount member. GB2324737 describes a device with an inflatable cuff for deflecting the epiglottis.

Laryngeal masks have several advantages over endotracheal tubes, which are longer and seal with the trachea below the vocal folds. One of the problems in laryngeal masks is of improving the seal made by the cuff with the patient's anatomy. In U.S. Pat. No. 4,995,388 there is described a laryngeal mask with an inflatable cuff and having a flexible collar secured to the cuff around the opening of the mask so as to locate in the tracheal opening. To be effective, however, the collar must have some rigidity so that it protrudes from the cuff. This can make it difficult to insert and position the mask without causing trauma.

### BRIEF SUMMARY OF THE INVENTION

It is an object of the present invention to provide an alternative laryngeal mask assembly.

According to the present invention there is provided a laryngeal mask assembly comprising a tube and an annular sealing cuff extending around the patient end of the tube, the cuff having a central opening that communicates with the bore of the tube, the assembly including an annular collar on the cuff extending around the opening and the collar being inflatable or deflatable.

The assembly may include a mount at the patient end of the tube, the cuff being mounted on the mount. The annular sealing cuff is preferably inflatable via an inflation line extending away from the cuff along the tube and the inflation line may extend in a groove formed in the outer surface of the tube. The collar and cuff are preferably inflatable or deflatable separately, such as via separate inflation lines. The collar may have a generally triangular section and when inflated preferably has a height of about 10 mm. The collar is preferably soft and floppy when not inflated. Alternatively, the collar may be naturally upstanding and be deformed to a more compact shape by the application of suction.

A laryngeal mask assembly according to the present invention will now be described, by way of example, with reference to the accompanying drawings.

### BRIEF DESCRIPTION OF THE DRAWINGS

FIG. 1 is a perspective view of the assembly from above;

FIG. 2 is a perspective view of the assembly from below; and

FIG. 3 is a cross-sectional side elevation view of the assembly.

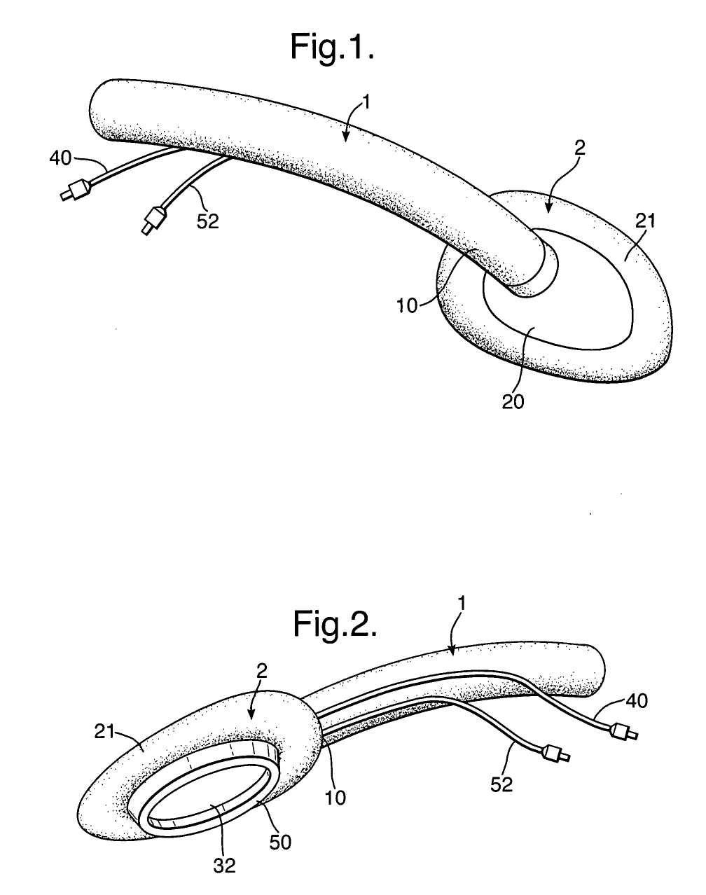

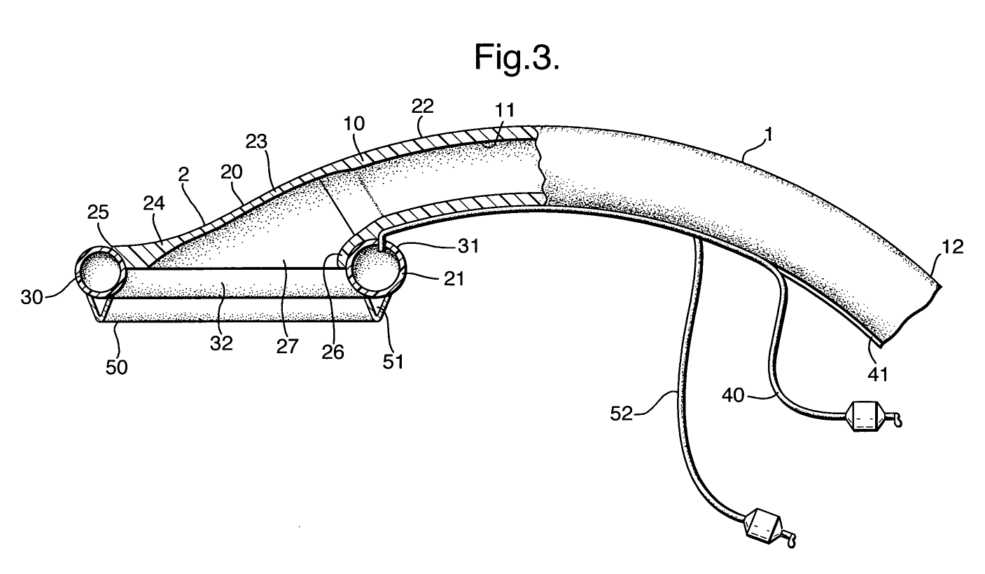

### DETAILED DESCRIPTION OF THE PREFERRED EMBODIMENT

The laryngeal mask assembly comprises a tube 1 and a mask formation 2 at the patient end 10 of the tube.

The tube 1 is of a bendable plastics material, such as PVC and is curved along its length. A bore 11 extends along the tube from its patient end 10 to its rear, machine end 12.

The mask 2 includes a mount 20 and an inflatable sealing cuff 21. The mount 20 is of a relatively stiff plastics material and is of generally shoe shape, as described in WO01/13980. The mount 20 is moulded integrally at the forward end of the tube 1, such as by injection moulding, to form an integral, single piece 22. The mount 20 tapers outwardly from its machine end 23 to its patient end 24, which is inclined to the axis of the machine end at an angle of about 25° so that the patient end of the mount has an oval shape with its forward end 25 being more pointed than its rear end 26. The patient end 24 of the mount 20 is inclined to face towards the inner side of the curve of the tube 1. Internally, the mount 20 has a cavity 27, which increases in cross-sectional area along its length, from the machine end.

The cuff 21 is tubular and of a thin flexible plastics material. The cuff 21 is formed into an annulus of the same shape as the patient end 24 of the mount 20 so that it is oval with its forwardly-directed end 30 being more pointed than its rearwardly-directed end 31. The cuff 21 encloses a central region or opening 32 of the same shape as the patient end 24 of the mount 20. The cuff 21 is attached around the patient end 24 of the mount 20 such as by means of an adhesive. The cuff 21 is inflated and deflated by means of an inflation line 40, which is provided by a separate small-bore tube communicating with the interior of the cuff and extending rearwardly along a groove 41 in the outside of the tube. When inflated, the cuff 21 seals with tissue in the region of the hypopharynx

The mount also includes a laryngeal collar 50 of annular shape and secured on the patient side of the cuff 21. The collar 50 extends around the central opening 32 of the cuff 21 and has the same shape as the cuff. The collar 50 is made of a thin flexible plastics material and has a hollow interior space 51 communicating with a collar inflation line 52. The nature of the material of the collar 50 is such that, when it is not inflated, it is relatively soft and floppy but, when inflated, it forms a relatively stiff upstanding, resilient annular wall about 10 mm high. The collar 50 has a triangular section, being wider towards its base than towards its apex. The collar 50 is arranged such that, when the mask is correctly inserted, the collar projects into the laryngeal inlet at the upper end of the trachea. It will be appreciated that increased ventilation gas pressure in the region of the opening 32 will deflect the collar 50 outwardly slightly so as better to seal with the tissue around the laryngeal inlet. When the collar 50 is deflated, however, it can easily be deformed as it is inserted or withdrawn from the patient. Thus, by making the collar 50 inflatable, it can have the best properties both for insertion/withdrawal and for sealing when in use.

Instead of having the collar inflatable separately from the cuff, the interior of the collar could communicate with the interior of the cuff so that both are inflated together. Separate inflation does, however, have the advantage that the pressures within the collar and cuff can be separately controlled.

The collar could be made of a stiffer material so that it was naturally upstanding or inflated and could be sucked down to a more compact shape by applying suction via the inflation line. A similar effect could be produced with a thin wall collar containing an open-cell foam. The foam would cause the collar to inflate itself and the collar could be compacted down by applying suction via the inflation line.

What I claim is:

1. A laryngeal mask assembly comprising: a tube having a bore extending along its length and having a patient end; an annular sealing cuff extending around the patient end of the tube, the cuff having a central opening that communicates with the bore of the tube; and an annular collar on said cuff extending around said opening, and wherein said collar is inflatable or deflatable.

2. A laryngeal mask assembly according to claim 1 including a mount at the patient end of said tube and wherein said cuff is mounted on said mount.

3. A laryngeal mask assembly according to claim 1, wherein said assembly includes an inflation line extending away from said cuff along said tube, and wherein said annular sealing cuff is inflatable via said inflation line.

4. A laryngeal mask assembly according to claim 3, wherein said tube has an outer surface and a groove formed along said outer surface, and wherein said inflation line extends in said groove.

5. A laryngeal mask assembly according to claim 1, wherein said collar and said cuff are inflatable or deflatable separately.

6. A laryngeal mask assembly according to claim 5, wherein said collar and said cuff connect with respective separate inflation lines.

7. A laryngeal mask assembly according to claim 1, wherein said collar has a generally triangular section.

8. A laryngeal mask assembly according to claim 1, wherein said collar when inflated has a height of about 10 mm

9. A laryngeal mask assembly according to claim 1, wherein said collar is soft and floppy when not inflated.

10. A laryngeal mask assembly according to claim 1, wherein said collar is naturally upstanding and is deformed to a more compact shape by the application of suction.

11. A laryngeal mask assembly comprising: a tube having a bore extending along its length and having a patient end; an inflatable annular sealing cuff extending around the patient end of the tube, the cuff having a central opening that communicates with the bore of the tube; an inflatable annular collar on said cuff extending around said opening; a first inflation line communicating with the interior of said sealing cuff by which said cuff can be inflated and deflated; and a second inflation line communicating with said collar by which said collar can be inflated and deflated.
